(19) United States
(12) Patent Application Publication
(10) Pub. No.: US 2002/0077581 A1
(43) Pub. Date: Jun. 20, 2002

(54) Simplified cerebral retroperfusion apparatus and method

(76) Inventors: Alan Davidner, Yorba Linda, CA (US); Thomas Kardos, Laguna Beach, CA (US)

Correspondence Address: G. DONALD WEBER, JR., Suite 850, 1038 N. Tustin Avenue, Orange, CA, 92867, US

(21) Appl. No.: 09/739,605

(22) Filed: Dec. 19, 2000

(51) Int. Cl. A61M 37/00; A61M 29/00; A61M 31/00

(52) U.S. Cl. 604/6.09; 604/5.01; 604/509; 604/102.01

(57) ABSTRACT

A method and apparatus for retroperfusing cerebral venous vasculature with autologous venous blood through a percutaneous entry site which incorporates a double-lumen catheter, a self-inflating pressure-regulated balloon mounted on the catheter and a perfusion outflow region either distal to the balloon for direct perfusion into the superior sagittal sinus, or proximal to the balloon for perfusion into the transverse sinus near the junction of the superior sagittal sinus.

### BACKGROUND

1. Field of the Invention

This invention relates to retroperfusion of ischemic cerebral tissues, in general, and to a method and apparatus using filtered, venous blood supplied through a single percutaneously-placed catheter and control apparatus related thereto, in particular, for performing the retroperfusion process.

2. Prior Art

Based on prior art, retroperfusion of cerebral ischemia has been successfully performed on animals and on at least one clinical trial. Prior equipment included a multiplicity of catheters and was sub-optimal due to reperfusion injury and the complexity of placing multiple catheters.

Frazee et al. (U.S. Pat. No. 5,908,407) and Frazee (U.S. Pat. No. 5,794,629) describe a complicated system requiring two or three catheters including an arterial catheter and two triple-lumen venous catheters.

Lundquist et al. (U.S. Pat. No. 5,011,468) also describes a complicated system with several catheters of triple-lumen design to supply arterial blood into a venous region of the brain. However, the methods and devices described by Frazee et al and/or Lundquist et al to date have failed to reduce the potential reperfusion injury which occurs when prolonged ischemic tissue is suddenly perfused with fully oxygenated blood, causing an oxygen blast and subsequent oxygen-free radical formation resulting in additional tissue necrosis beyond that caused during the ischemic period.

Wakida et al. (Short-term synchronized retroperfusion before reperfusion reduces infarct size after prolonged ischemia in dogs. 1993 Nov.) and. Hatori et al. (Short-term treatment with synchronized coronary venous retroperfusion before full reperfusion significantly reduces myocardial infarct size. American Heart Journal, 1992 May.) have demonstrated that retroperfusion before fully oxygenated reperfusion can reduce reperfusion injury and coronary infarct size.

Uriuda et al. (Effects of superoxide dismutase administered by coronary sinus retroperfusion on ischemic reperfused canine heart. Journal of the Japanese Association for Thoracic Surgery, 1993 Jan.) has demonstrated that oxygen-free radical formation does play a role in causing reperfusion injury, at least in coronary situations.

Mohl et al. (U.S. Pat. No. 4,934,996: Pressure-controlled intermittent coronary sinus occlusion apparatus and method) and Feindel et al. (The effectiveness of various modes of nonsynchronized retrovenous perfusion in salvage of ischemic myocardium in the pig. Canadian Journal of Cardiology, 1991 Oct.) have demonstrated that venous blood has sufficient oxygenation to reduce coronary ischemia. However, this technique has not been used in stroke-type situations.

Thus, in the past, cerebral retroperfusion has been impractical due to the multiplicity of catheters required and/or insufficient in effectiveness due to the short time window under which the technique must be applied when reperfusion injury is not mitigated.

Co-pending U.S. patent application Ser. No. 09/152,528; SEPTICEMIA PREVENTION AND TREATMENT SYSTEM by Davidner et al describes a blood treatment and filtration system which can be utilized in conjunction with the catheter system of the instant invention to provide additional treatment benefits for a stroke patient.

### SUMMARY OF THE INSTANT INVENTION

This invention relates to a method and apparatus for treatment of ischemic stroke via a single catheter selectively inserted into the superior sagittal sinus or a transverse sinus and an extracorporeal cytokine filtering pump system. The catheter contains two lumens and, in one embodiment, an inflatable balloon adjacent to the distal end thereof. One lumen, the perfusion lumen, is used for introducing pressurized blood into the ischemic area. When utilized, the balloon is located at the distal end of the catheter and is in communication with the perfusion lumen. The second lumen is used for monitoring pressure of the blood at the distal end of the catheter or adjacent to the balloon. The pressurized venous blood is derived from the introducer sheath of the same catheter and is pumped from the side-port of the catheter introducer into the perfusion lumen to provide venous blood under pressure for retrograde delivery into the superior sagittal sinus and to inflate the balloon with limited pressure. The venous blood from the side-port of the catheter is also hemodiluted, filtered for cytokines, cell mediators and neutrophils, and then hemoconcentrated back to substantially the original hematocrit in an extracorporeal circuit.

Preferably, the autologous venous blood is returned hypothermic to reduce the oxygen demand of the ischemic tissues. Thus, a cooling device may be utilized to cool the blood in the extracorporeal circuit prior to reintroduction of the blood to the patient.

The extracorporeal system may incorporate a blood oxygenator to gradually increase the oxygen content of the venous blood for gradually increasing tissue oxygenation. Likewise, negatively-charged platinum electrode may be included in the extracorporeal circuit to remove positively charged ions which can occur during ischemia.

An object of the present invention is to perform all functions required for cerebral retroperfusion in a double-lumen venous catheter, and to increase the time window available for patient treatment by mitigating reperfusion injury with the use of cytokine filtering. The use of partially oxygenated venous blood and the removal of positive calcium ions using a platinum electrode are additional features of the invention.

### BRIEF DESCRIPTION OF THE DRAWINGS

FIG. 1 shows one embodiment of a system of the instant invention which includes primary and secondary extracorporeal fluid circuits.

FIG. 2 is a block diagram of the electronic control system for the system shown in FIG. 1.

FIG. 3 shows one embodiment of a superior sagittal sinus catheter insertion in accordance with the instant invention.

FIG. 4 shows one embodiment of a transverse sinus catheter insertion in accordance with the instant invention.

FIG. 5 shows another embodiment of a superior sagittal sinus catheter insertion in accordance with the instant invention.

FIGS. 6, 6A and 6B show one embodiment of the catheter tip configuration used with the catheter placement shown in FIG. 3.

FIGS. 7, 7A and 7B show one embodiment of the catheter tip configuration used with the catheter placement shown in FIG. 4.

FIG. 8, 8A and 8B show one embodiment of the catheter tip configuration used with the catheter placement shown in FIG. 5.

### DESCRIPTION OF A PREFERRED EMBODIMENT

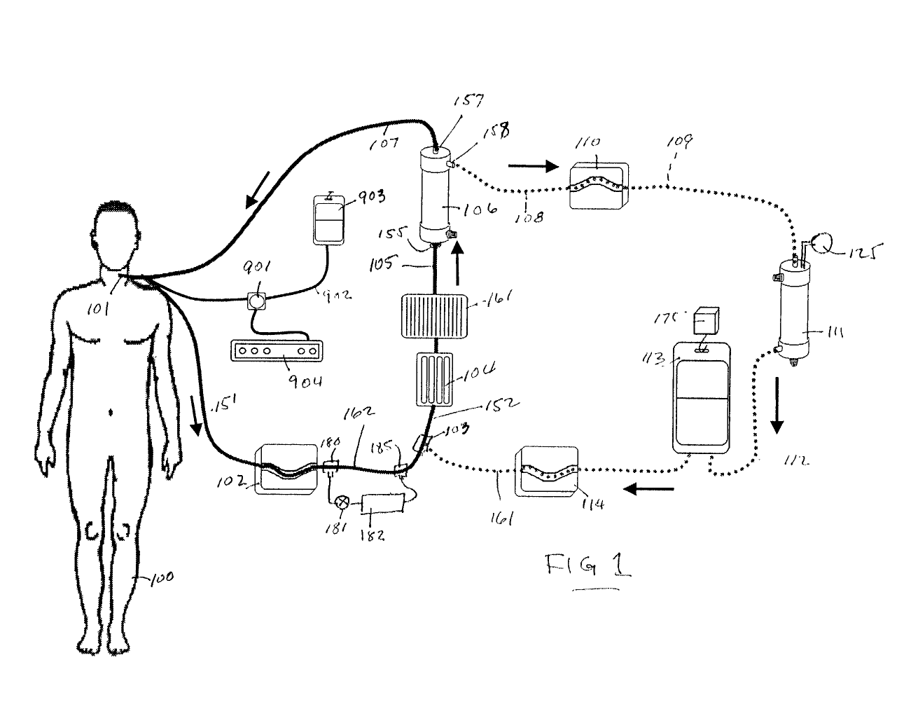

Referring now to FIG. 1, there is shown one embodiment of the system defined in the instant invention. The primary circuit (shown in solid line) is an extracorporeal blood fluid path which is operative on the blood of the patient, whereas the secondary circuit (shown in dashed line) is an extracorporeal fluid path which is operative on the saline diluent and ultrafiltrate which has been filtered out of the fluid in the primary circuit. The system comprises a plurality of components, typically, connected by standard medical extracorporeal tubing and connectors. As such, the system may be attached to a patient 100 via cannulation 101 or it may be incorporated into extracorporeal circuitry already serving a patient such as in hemodialysis or cardiopulmonary bypass. The cannulation may be performed on either left or right internal jugular veins at the neck, or either left or right femoral veins at the thigh. The preferred insertion site is the right internal jugular vein (followed in preference by the right femoral vein) for most direct entry into the superior sagittal sinus.

As represented herein, the circuitry resembles that of modern hemodialysis in terms of vascular access, bypass mode (venovenous or arteriovenous), blood flow rate, the use of a hemoconcentrator and duration of application. Therefore, some of the components described herein could be incorporated into a hemodialysis system, as well.

As noted, FIG. 1 shows one embodiment of the system of the instant invention, including several alternative configurations. Thus, blood from the patient 100 enters the tubing 151 via a suitable connector such as a venous cannula 101 (described in detail infra) of sufficient diameter to permit drainage flow of whole blood up to about 50-400 ml/min.

The patient's venous blood proceeds via polyvinyl chloride (PVC) or other suitable tubing 151 to a pump 102, which can be a positive displacement, a centrifugal pump, or the like, which regulates flow at about 50-400 ml/min through the system.

As blood from pump 102 has alternative routes. In the alternate, optional route, blood flows through a diverter 180 wherein at least a portion of the blood is supplied to clamp 181 (or other selective flow blocker) which is selectively opened. Blood, which passes through clamp 181, is supplied to filter 182 which filters neutrophils from the blood. Blood from filter 181 is returned to tubing 162 through a suitable connector 185.

Also, as shown in FIG. 1, the primary path for blood from pump 102 passes through tubing 162 to a suitable connector 103 where the blood mixes with a suitable isotonic diluent, such as plasmalyte or a saline solution. A variety of such solutions, referred to as “crystalloids”, are available in the art. The diluent is supplied from diluent source 113 which, typically, comprises a large capacity reservoir for storing an admixture of reclaimed (or converted) ultrafiltrate. The diluent is delivered to connector 103 via pump 114 which can be a roller pump or the like. The diluent is supplied at a flow rate which when mixed with the blood from conduit 151 results in a hematocrit of about 50% of normal. For example, a patient with a hematocrit of 30% is reduced to a hematocrit of 15%.

The diluted venous blood then passes through tubing 152. As shown in FIG. 1, the blood is supplied to the optional oxygenator device 104 and/or the optional heat exchanger 161 as described in detail infra. That is, the oxygenator 104 and/or the heat exchanger 161 can provide advantageous operation of the system but are not required in order to practice the invention.

The diluted blood passes through tubing 105 (and optional oxygenator 104 and optional heat exchanger 161, if utilized) to hemoconcentrator 106 which has approximately 1.2 to 2.4 m² exchange surface with pore size of about 70-120 kD, although these parameters are not limitative of the invention, per se. Thus, some of the target molecules are effectively removed from the fluid flowing therethrough by sieving to down regulate the immune response to mitigate endothelium and tissue damage due to an over reactive immune response. The hemoconcentrator 106 (described in copending application Ser. No. 09/152,528 noted above) is preferably oriented vertically so that blood flows from bottom to top. This arrangement aids in priming and debubbling the system with crystalloid diluent before blood enters the circuit.

The blood leaves the hemoconcentrator 106 at port 157 at a hematocrit approximating its entry into the system and returns to the patient via tubing 107 and cannulation 101 and catheter as described in detail infra.

The pressure across the hemoconcentrator 106 is monitored at sample ports and decreases by about 50 mmHg or more from inlet 155 port to outlet 157 port at a combined blood and diluent flow of about 100-800 ml/min and blood hematocrit of about 50% of normal. Flowing through the hemoconcentrator, the blood hematocrit is increased from about 50% of normal back to about 100% of normal.

The material filtered from the blood (i.e. ultrafiltrate) by hemoconcentrator 106 is removed via tubing 108 connected to the outlet port 158. Hemoconcentrator pump 110 (such as a roller pump or the like) propels the ultrafiltrate from hemoconcentrator 106 through tubing 109, to filter 111 which is, typically, constructed and designed for the removal of, inter alia cytokines.

Irrespective of the specific construction thereof, filter 111 preferably exhibits a porosity of about 10 kD, for example, and about 99% of the unwanted cytokines are captured in filter 111 which is disposed of at the termination of the procedure.## Thus, the majority of the smaller molecules pass through filter 111 and tubing 112 to the large-capacity diluent reservoir 113 and mix with the crystalloid diluent. Pump 114 propels the admixture of crystalloid and filtrate from the secondary circuit back to the primary circuit via Y-connector 103 as described supra. Thus, smaller molecules and diluent can be conserved by passage thereof completely through the secondary circuit, while plasma proteins and other large molecules are conserved by retention thereof in the primary circuit at the hemoconcentrator.

Pump flow is based on the venous sinus pressure at the tip of the catheter 514 and the hematocrit of the patient 100, and can be regulated with knowledge of sinus pressure and hemoconcentrator inlet pressure as determined in a conventional manner by transducer 902 connected to catheter lumen 307 and to inlet port 155, respectively. Port 155 may include a stopcock for monitoring the inlet pressure and/or for sampling of the fluid.

In one embodiment, a single pump 102, positioned as shown, regulates patient blood and diluent flows using either a pump with the capability of dual raceway control or a traditional single raceway pump. In the latter case, the pump flow would be regulated at 100-800 ml/min, accounting for the combined flows from the patient 100 and the diluent reservoir 113.

In this embodiment, the blood is, typically, diluted on a one-to-one (1:1) ratio with a saline solution then hemoconcentrated using the 70-90 kilo-Dalton (kD) filter 106 as shown in FIG. 1. The ultrafiltrate is filtered for cytokines using the 10 kD filter 111 as shown. The volume of diluent in reservoir 113 (which may include a recirculation bag) is maintained substantially level over time by measuring the weight of the diluent volume in the reservoir 113 using the load cell 175, and adjusting the ratio of flows in the hemoconcentrator pump 110 and the diluent pump 114, as required. If the diluent level in the reservoir 113 drops, the flow rate of hemoconcentrator pump 110 is increased in relationship to the flow rate of diluent pump 114. Conversely, if the diluent level increases, the flow rate of hemoconcentrator 110 is reduced in relationship to the diluent pump flow rate. The ratio of the flow rates of the blood pump 102 and the diluent pump 114 is maintained at substantially one-to-one, which provides for a substantially 50% dilution of the blood from patient 100.

As related to retroperfusion, the filtering of cytokines from the blood by filter 111 can improve the patient outcome by removing from the blood by products that are elevated during ischemia, and thereby reducing reperfusion injury.

Venous blood is utilized in this system because normal venous blood has greater oxygenation in comparison to ischemic tissues, but lesser oxygenation compared to arterial blood. Thus, venous blood imparts oxygen to the ischemic tissues while minimizing oxygen shock and, thereby, reperfusion injury which occurs when tissue which has been ischemic for a prolonged period of time is suddenly reperfused with fully oxygenated arterial blood. Free oxygen radicals (molecules with an odd number of oxygen atoms and a reactive electron) are produced during an oxygen shock of ischemic tissues. These radicals chemically react with surrounding proteins and cause tissue necrosis. By initially reperfusing such tissues with venous blood which has a lower oxygen content than arterial blood, this chemical cascade is reduced whereby some of the ischemic tissue can be restored to normal function.

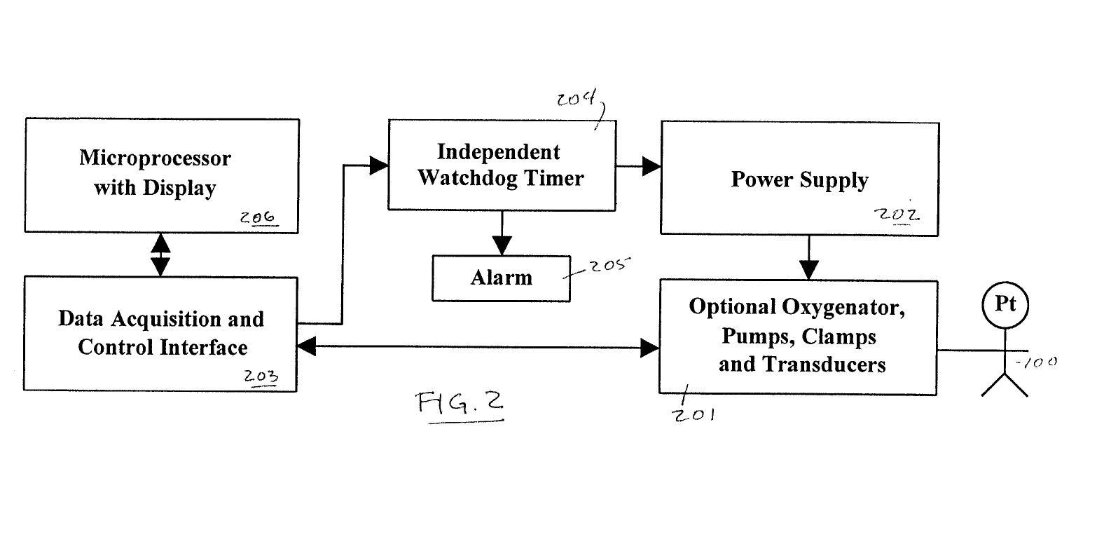

In addition, venous blood reduces the complexity of the retroperfusion system and avoids the need for a separate catheter for harvesting the patient's arterial blood as required in the prior art. Thus, the side port of the introducer sheath of the single catheter (see FIG. 3) is employed as the cannulation to harvest venous blood, thereby reducing the number of catheters needed and the risk of infections to the patient from multiple catheter entry points.

As described supra, venous blood is utilized in part to mitigate the effects of an oxygen blast at the initial reperfusion of ischemic tissues in order to mitigate reperfusion injury. Once the ischemic tissue has been prepared to safely accept a more complete reperfusion by the mild oxygenation with retroperfused venous blood, the mild cooling of the ischemic region to reduce oxygen demand, washing out of harmful metabolites and byproducts of ischemia and cytokine filtering, the critical period for reperfusion injury has been averted. Thereafter, in some applications, the oxygen content of the retroperfused blood may safely be raised to maximize tissue oxygenation. To this effect, oxygenator 104 is provided as an optional apparatus to gradually increase the oxygen content of the retroperfusate supplied to patient 100 via tube 107 following the initial tissue preparation. If oxygenation is not desired, the oxygenerator 104 can be omitted from the system.

To provide a more comprehensive approach to the mitigation of the various causes of reperfusion injury, additional apparatus and a method is provided to remove positively-charged Calcium ions (Ca++) which are often proliferated during ischemia by a chemical cascade in ischemic tissues and contribute to tissue necrosis. In a preferred embodiment, a substantially non-corrosive and non-reactive platinum electrode 125 is provided in the extracorporal circuit as part of, or adjacent to, the cytokine filter 1 1 1. The electrode 125 is charged negatively with respect to the body of patient 100 by a voltage in the range 1-10 VDC. This voltage potential creates a voltage gradient across the extracorporal fluid path that provides blood which has been charged negatively at the tip of the retroperfusion catheter within the patient (described infra) with respect to the rest of the body of patient 100. This negatively charged retroperfusate attracts the positively charged CA++ ions in the vicinity of the ischemic area, drawing them into the venous blood flowing in the cerebral venous vasculature and the extracorporeal paths. With the voltage gradient being most negative at the platinum electrode 125 in the extracorporeal circuit, the ions are moved and concentrated near the electrode and are removed from the blood at filter 111. Moreover, because electrode 125 is located at or near the cytokine filter 111, CA++ ions which are removed from the patient's blood at the hemoconcentrator 11 1, travel within the ultrafiltrate fluid toward the platinum electrode 125, where the concentration thereof can be increased or decreased depending on the voltage level on the electrode.

By cooling of the blood in the extracorporeal circuit by 1-5° C., the returned blood will mildly cool the ischemic tissues which it is targeted toward. This technique adds another measure of protection to these tissues by reducing the oxygen demand thereof, and, therefore, reducing necrosis in the absence of sufficient oxygenation. Cooling of the blood can be accomplished as a result of ambient or natural cooling. Alternatively, the optional heat exchanger 161 can be used to achieve the desired temperature adjustment, if desired. (Of course, heating of the blood can be accomplished as well, if so desired.) The flow rates provided by this system are in the range of 50 to 400 ml/min, as needed, to generate cerebral venous pressures in the range of 10 to 25 mmHg at the entrance to the super sagittal sinus (see infra) for effectively reducing tissue ischemia while maintaining a safe cerebral venous pressure. Pressures substantially greater than 25 mmHg may cause unwanted adverse effects such as cerebral venous congestion, unsafe intracranial pressures and cerebral hemorrhage. The system is provided with a conventional pressure sensing apparatus (see infra) which is monitored and provides for alarming and/or automatically stopping or reducing the retroperfusion flow if the safe venous pressure range is exceeded.

Referring now to FIG. 2, there is shown a block diagram of the electronic controls for the system of the instant invention. The diagram includes a patient 100 (similar to the patient 100 in FIG. 1) who is connected to the extracorporeal fluid circuits of the type described relative to FIG. 1. The electronic controls include multiple electronic circuits and subsystems including the components described in detail infra.

The components in circuit 201 receive power from the power supply 202. The data acquisition and control interface 203 exchanges signals with the circuit 201. The interface 203 also supplies input signals to the independent “watchdog” timer 204. The timer also supplies control signals to the power supply 202 and to an alarm 205. A microprocessor 206 is connected to the control interface 203 to exchange information therebetween.

In operation, the system operator inputs appropriate information into the microprocessor 206, for example by an interactive touch screen or the like.

The microprocessor 206 also receives the appropriate operational information such as pump speeds, pressures and temperatures from the data acquisition and control interface 203. The microprocessor supplies appropriate information such as pump and clamp voltages to operate the electromechanical devices in the extracorporeal fluid circuits. This information is supplied to the circuit 201 (and the discrete components thereof) for controlling the operation of the pumps and clamps of the extracorporeal fluid circuits relative to procedures used with regard to the blood of patient 100.

The timer 204 receives inputs from interface 203 to monitor the state of operation of the computer 206 and the interface 203, for example, through an oscillating digital signal. If the frequency of oscillation of this signal is less than or greater than preset limits, the watchdog timer 204 interprets this as inappropriate operation, and acts to stop the function of pumps and clamps by turning off the power thereto. The timer 204 operates to selectively enable and disable the power supply 202 and, thereby, control the operation of the circuits 201 to ensure proper functionality thereof. In addition, timer 204 selectively activates alarm 205, when appropriate, to indicate that a malfunction has been detected.

Referring now to FIG. 3, there is shown a simplified representation of the cerebral vessel structure of a patient 100 taken from the front. It should be understood that many other veins and vessels which exist in the vicinity of the torcula junction 310 have been omitted for convenience. Thus, one embodiment of the invention showing catheter 316 within the simplified representation of the vessels will be more easily understood.

In a manner commonly used for the placement of catheters, the procedure of the present invention begins when a percutaneous entry 320 is made through the skin and into the internal jugular vein 301 of the patient 100 with the introducer 309. A guidewire (not shown) is urged through the introducer and along the desired venous path to its final operative position suggested in FIG. 3. In this position, the guidewire extends through the right sigmoid sinus 302, the right transverse sinus 303, the torcula 310, and into the superior sagittal sinus 305. Placement of the guidewire is conventional and is facilitated by its nimble structure which enables it to be moved along a circuitous path.

It is conventional knowledge that the path into the superior sagittal sinus is most direct from the right transverse sinus, right sigmoid sinus, and right internal jugular vein. Therefore, advancement of a guidewire into the superior sagittal sinus for most cases is most suitable from a right internal jugular vein entry point, or with a longer guidewire and catheter, from the right femoral vein entry point. In the latter case (not specifically shown herein) the wire (and subsequently the catheter) are advanced up through the inferior vena cava, through the right atrium and through the superior vena cava to the right internal jugular vein and beyond as stated supra.

With the guidewire properly positioned, the distal end of the catheter 316 is threaded over the proximal end of the guidewire. With the guidewire dictating the preferred path, the catheter 316 is pushed along the guidewire until it reaches the operative position illustrated in FIG. 3. In this position, the balloon 341 at the distal end of catheter 316 is disposed in the superior sagittal sinus 305 and slightly beyond the torcula junction 310.

The blood flow lumen 306 is connected to the system shown in FIG. 1, in particular, to tubing 107 to receive the venous blood flow form the hemoconcentrator 106. The pressure lumen 307 is connected to transducer 902 as shown in FIG. 1. In addition, the side-port 308 of the introducer 309 is operative to harvest the patient's own venous blood and to supply this blood to the treatment system via tubing 151. Thus, the patient's autologous, venous blood which has been filtered, cooled and partially oxygenated, is retroperfused into the ischemic area under treatment.

When operatively disposed, the balloon 341 can be inflated to at least partially occlude the superior sagittal sinus 305. This effectively isolates the venous torcula junction 310 and the superior sagittal sinus 305 with the operative region of the catheter 316 disposed in the superior sagittal sinus 305. Having at least partially inflated the balloon 341 by initiating forward flow of blood through lumen 306 in catheter 310, for example, by operation of pump 102 (in FIG. 1), perfusion of the superior sagittal sinus 305 can begin. After inflating the balloon 341, the venous blood will continue to flow out the tip 315 of the catheter 316, thus perfusing the superior sagittal sinus 305 and the adjacent ischemic area.

As the perfusion procedure begins, blood flow begins in the superior sagittal sinus 305 in a retrograde direction illustrated by the arrow 363. This retrograde flow perfuses the capillary bed which drains into the superior sagittal sinus 305 from the cerebral cortex in both the left and right hemispheres through smaller veins connecting to the superior sagittal sinus 305. A preferred range for the retrograde flow rate is between 50 and 400 milliliters per minute as required to raise the pressure in the superior sagittal sinus in the range of at least 10 mmHg but not over about 30 mmHg.

Pressure adjustments to control this pressure and flow rate can be made at the blood pump 102 in FIG. 1. If the balloon 341 is deflated, the degree of occlusion is reduced, thereby permitting increased antegrade flow in the transverse sinus 303 As this antegrade flow is increased, the pressure in the junction 310 is reduced along with the pressure and retrograde flow in the superior sagittal sinus 305.

When the retroperfusion procedure is completed, the flow of oxygenated blood into the perfusion lumen 301 can be stopped, and the balloon 341 can be deflated. Balloon deflation can be achieved by reversing flow in the perfusion lumen through connection 107 by operating pump 110 forward or by operating pump 102 in reverse. With the balloon deflated and the catheter 316 in a low profile state, it can be withdrawn through the introducer 309.

Referring now to FIG. 4, there is shown another embodiment of the operation of the catheter 416 within the cerebral vessels. Again, it will be understood that may other veins which exist in the vicinity of the junction 410 have been omitted for convenience. In this procedure, the introduction of a guidewire (not shown) is through a percutaneous puncture 420 in the internal jugular vein 401 or the femoral vein as discussed supra, and the guidewire is urged along the desired venous path to its final operative position suggested in FIG. 4. In this position, the guidewire extends through the right sigmoid sinus 402, through the right transverse sinus 403, through the junction 410 and into the left transverse sinus 404 until it reaches the operative position illustrated in FIG. 4. In this position, the balloon 416 is disposed in the right transverse sinus 404 and past the junction 410. It should be understood, of course, that this procedure (as well as in FIG. 3) can be carried out in the opposite direction wherein the guidewire passes through the left sagittal sinus and so forth, so that the balloon is ultimately disposed in the right transverse sinus.

When operatively disposed, the balloon 441 can be inflated to at least partially occlude the left transverse sinus 404. This effectively isolates the venous torcula junction 410 and the superior sagittal sinus 405 with the operative region 490 of the catheter 416 disposed slightly beyond the torcula junction 410. That is, the operative region 490 of the catheter 416 includes one or more apertures therein which communicate with the blood lumen 406. Having at least partially inflated the balloon 441 by pressurizing thereof, perfusion of the superior sagittal sinus 405 can begin. That is, pressurized blood can exit the catheter into the superior sagittal sinus 405.

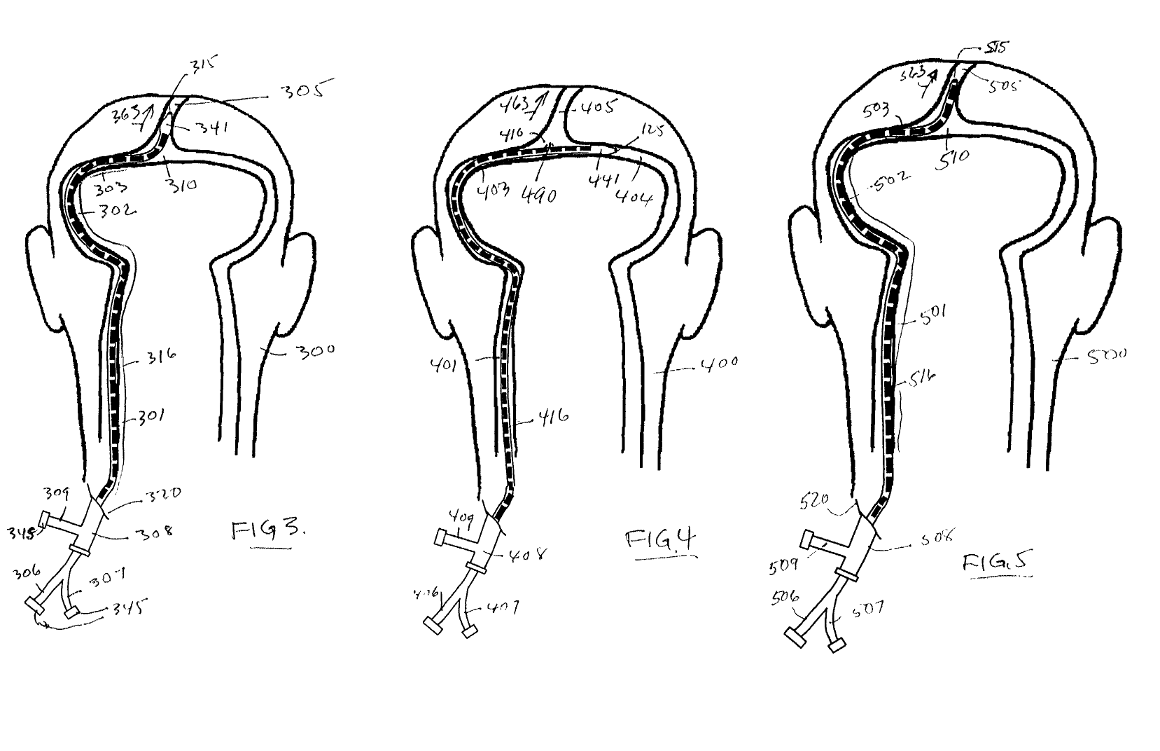

Autologous blood is supplied to the catheter 416 in the same manner described relative to FIG. 3 through the associated blood lumen 406 to exit the catheter 416 through the apertures or ports (as described relative to FIG. 6) in the torcula junction 310.

At this point, it is apparent that the oxygenated blood under pressure in the lumen 406 can also escape through the distal hole 415 in catheter 416. However, provision is made for the majority of blood flow to exit the catheter 416 in the operative region 490 located proximal to the balloon as described infra.

As the perfusion of oxygenated blood begins, the junction 410 is pressurized to the extent that blood flow begins in the superior sagittal sinus 405 in a retrograde direction illustrated by the arrow 463. This retrograde flow perfuses the capillary bed which drains into the superior sagittal sinus from the cerebral cortex in both the left and right hemispheres through smaller veins connecting to the superior sagittal sinus. Again a preferred range for this retrograde flow rate is between 50 and 400 milliliters per minute, as required to increase venous sinus pressures in the range of about 10 to 30 mm Hg.

Pressure and flow rate adjustments at junction 410 are effectively controlled by the blood pump 102 shown in FIG. 1. Minor adjustments in pressure can be made as described supra inflating and deflating the balloon 416 to adjust the degree of occlusion in the transverse sinus 404. If pump 102 speed is reduced, the flow rate and pressure at junction 410 are reduced and vice versa. As flow is reduced or reversed by operating pump 1 10 forward or pump 102 in reverse, the balloon 416 is deflated, thereby permitting increased antegrade flow in the transverse sinus 404. As this antegrade flow is increased, the pressure in the junction 410 is reduced along with the retrograde flow in the superior sagittal sinus 405.

When the retroperfusion procedure is completed, the flow of oxygenated blood into the perfusion lumen 406 can be stopped, and the balloon 416 can be deflated and withdrawn through the puncture site in the internal jugular or femoral vein as described supra.

Referring now to FIG. 5, there is shown another embodiment of the invention using the two-lumen catheter 516 within the simplified representation of the cranium of a patient. Again, the catheter 516 may be formed from polyurethane. It will be understood that other veins and vessels exist in the vicinity of the torcula junction 510 as in the representations in FIGS. 3 and 4.

Again, the procedure of this embodiment of the present invention begins with the introduction of a guidewire through the introducer 508 and a percutaneous puncture 520 made through the skin and into the internal jugular vein 501 (or femoral vein) of the patient 500, along the desired venous path through the right sigmoid sinus 502, the right transverse sinus 503, the torcula 510, and into the superior sagittal sinus 505.

The catheter 516 is threaded over the proximal end of the guidewire. With the guidewire dictating the preferred path, the catheter 516 is pushed along the guidewire until it reaches the operative position illustrated in FIG. 5. In this position, the distal end of the catheter, similar to the catheters described supra but without a balloon thereon, is disposed in the superior sagittal sinus 505. By appropriate selection, the catheter will at least partially occlude the superior sagittal sinus 505. This partially isolates the venous torcula junction 510 and the superior sagittal sinus 505 with the operative region of the catheter 516 disposed within the superior sagittal sinus 505. Upon initiating forward flow through lumen 506, perfusion of the superior sagittal sinus 505 can begin.

As in the other embodiment, autologous oxygenated blood can be taken from any venous source in the body. In the preferred embodiment, the blood lumen 506 is connected to the source as discussed with reference to FIG. 1. This blood is introduced into the connector blood lumen 506 and passes through the catheter to exit through the distal port 515.

As the perfusion procedure begins, blood flow begins in the superior sagittal sinus 505 in a retrograde direction illustrated by the arrow 563. This retrograde flow perfuses the capillary bed which drains into the superior sagittal sinus from the cerebral cortex in both the left and right hemispheres through smaller veins connecting to the superior sagittal sinus. Once again, the preferred range for the retrograde flow rate is between 50 and 400 milliliters per minute, as required to increase sinus pressures within the range of about 10 to 30 mmHg. In this embodiment, the catheter tip without balloon is relied on to partially occlude the superior sagittal sinus, a greater flow within the 50 to 400 ml/min range is required to raise sinus pressure within the target range of 10 to 30 mmHg than in the prior embodiments with a balloon. However, in some patients where the size of the vasculature is sufficiently small in relationship to the diameter of the catheter, this embodiment without a balloon may be more appropriate and may actually provide greater safety in preventing excessive pressure in the superior sagittal sinus 505.

Adjustments to control this pressure and flow rate can be made at the blood pump 102 in FIG. 1. The degree of pressure and flow is selected to provide retrograde flow or antegrade flow in the superior sagittal sinus 505. In this embodiment, antegrade flow is not prevented in the transverse sinus 503.

When the retroperfusion procedure is completed, the flow of oxygenated blood into the perfusion lumen 506 can be stopped, and the catheter 516 can be withdrawn from the port 509.

Referring now to FIGS. 6, 6A and 6B, there are shown external and cross sectional representations of the catheter 600 tip used with the instant invention to provide a superior sagittal sinus insertion as shown in FIG. 3.

The catheter 650 is a double lumen tube with an outer diameter of approximately 4-8 French, although this dimension is not limitative of the invention. The catheter includes a small, oval pressure lumen 610 of approximately 0.03 inches in diameter and a large semi-circular blood flow lumen 611 that comprises a substantial portion of the remainder of the cross sectional area of the catheter. A balloon 616 formed of flaccid material, as is conventional in the art, is attached to the catheter at the proximal end thereof. The catheter has an overall taper that decreases in diameter from the proximal end (with the luer fittings or the like thereat as suggested in FIG. 3) toward the distal end with the balloon.

One change in diameter occurs at the proximal end of the catheter 650 where a substantially larger than 8 French outer diameter exists to allow connection of access tubes into the two lumens (see FIG. 3). Preferably, the access tubes terminate at luer locks 345 or the like which enable connection to the pressure and blood lumens. The two luer locks connecting to the two lumens are of appropriate polarity or shape to prevent misconnection of the blood lumen to the pressure lumen and vice versa.

Another major change in diameter occurs at the distal end of the catheter 650 where the diameter is reduced to permit bonding thereto of a circumferential balloon 616 which, when deflated, permits the overall diameter of the catheter and the balloon to be substantially the same as the diameter of the catheter just proximal to where the balloon is attached.

The catheter 650 is inserted into a vein using a conventional percutaneous introducer sheath 309 that has a diameter at least 2 French sizes greater than the outer diameter of the catheter. This arrangement permits harvesting of the patient's venous blood through the side port 309 of the introducer at the target flow rates of up to 400 ml/min without risking significant hemolysis.

In one embodiment of the catheter 650, the primary blood exit port 615 is located at the distal tip of the catheter. The exit port has an opening in the range of 80% to 95% of the cross section of the blood lumen 611. This cross section area is less than 100% of the total cross section in order to provide a small restriction in the outflow of blood thereby to force some blood through at least one of orifices 612 and 613 to inflate the balloon 616. That is, the orifices 612 and 613 pass through the catheter but are disposed within the balloon 616.

It is seen that the ratio of hole sizes of orifices 612, 613 and 615 permits adjusting the balloon inflation pressure for any given blood flow rate. For example, the greater the diameter of orifice 612 in relation to the diameter of orifice 613, the greater the inflation pressure of the balloon. In addition, the smaller the diameter of orifice 615 alone and relative to the other orifices, the greater the inflation pressure of the balloon 616. The converse is also true in both instances. Thus, the balloon inflation pressure can be controlled by adjusting the diameter of orifices 612, 613 and 615 as desired. Preferably, the orifices 612 and 613 are located substantially at opposite sides of the catheter 650 shaft as shown in FIG. 6B in order to reduce blood stagnation in the balloon by increasing the turbulence with which the blood flows into and out from the balloon between these two orifices.

In a preferred embodiment of the catheter, the pressure lumen 610 is open to the outside of the catheter via aperture 614 at a point between the balloon 616 and proximal to the catheter tip 615. Thus, the measurement of pressure within the venous vessel is made distal to the balloon. The orifice 614 is, typically, spaced from the tip of the catheter by between 1-5 millimeters. This spacing avoids tip-vertex artifacts caused by the high speed of the ejected blood and thereby reduces errors in the pressure reading.

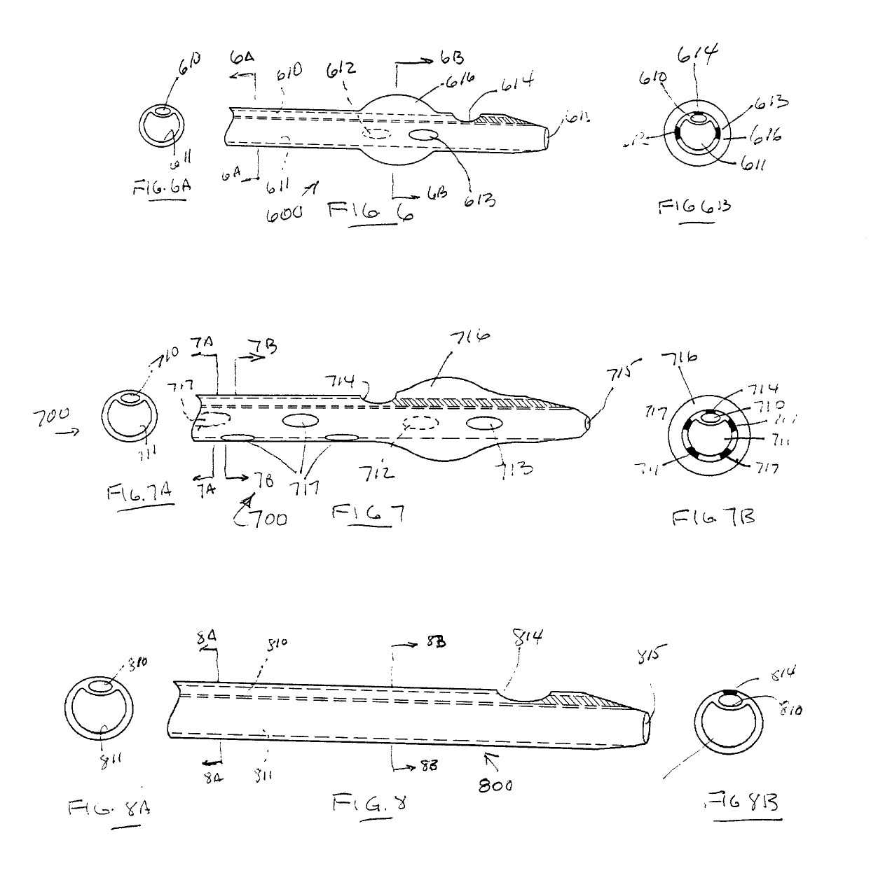

Referring concurrently now to FIGS. 7, 7A and 7B, there are shown an external and cross sectional representation of the catheter tip 700 used with the instant invention to provide blood flow to the superior sagittal sinus by a left transverse sinus insertion as shown in FIG. 4. In this embodiment, the catheter 700 includes lumens 710 and 711 (similar to the lumens in catheter 600 described supra). In catheter 700, the location of the pressure orifice 714 and the primary exit 715 of blood from the blood lumen 711 of the catheter are on opposite sides of the balloon 716 as shown in FIG. 7. The location of the tip of this catheter is such that the balloon is located past the confluence 410 of the sinuses (see FIG. 4), whereby the blood is ejected from the catheter 700 towards the superior sagittal sinus 405 (see FIG. 4) through the orifices 717. That is, balloon 716 occludes the left transverse sinus Perforce, the catheter 700 partially occludes the right transverse sinus as shown in FIG. 4 (or the left transverse sinus if the opposite direction insertion is utilized), thereby providing some resistance to the normal venous drainage and forcing inducing at least some of the blood ejected through orifices 717 into the superior sagittal sinus 405 especially in view of the position of the orifices 717 relative to the superior sagittal sinus.

To avoid stagnation of the blood in the balloon in this embodiment, a small orifice 715 is provided at the distal tip of the catheter. Normally, the opening of orifice 715 is in the range of 5% to 20% of the cross section of the blood lumen 711. This arrangement permits blood to enter the balloon at orifice 712 and flow out of the balloon at orifice 713 before exiting the catheter at orifice 715. Thus, the balloon 716 can be inflated, but stagnation is avoided as in the case of balloon 616 in FIG. 6.

The pressure lumen orifice 714 in this embodiment is proximal to the balloon 716 to permit sensing of pressure in the same region of the venous sinus where the ejected blood is delivered. As shown, the orifice 714 is located at a different region around the circumference of the catheter than orifices 717 in order to reduce errors in the pressure reading which could be adversely affected by the jets of blood exiting orifices 717. In addition, the orifices 617 are, preferably, arranged equidistantly around the catheter circumference in order to distribute pressure from the exiting jets of blood.

In the embodiments of the catheter shown in FIGS. 6 and 7, the tube is, typically, formed from polyurethane. The balloon can also be formed from polyurethane or some other elastomeric, compliant material. It will be apparent to those skilled in the art that many other types of materials will be more or less suited for a particular procedure. The balloon may be considered to be a non-elastic bladder, whereby it will fill to the substantially fully inflated diameter with low pressure from low flow past orifices such as apertures 612 and 712, and will not substantially inflate further to any greater diameter with additional pressure from a higher flow.

The pressure monitoring in the system is achieved via a procedure described relative to concurrent reference to all of the Figures. The pressure monitoring uses a conventional fluid column extending from port 307 through lumen 610 and out through orifice 614 in the preferred embodiment relative to FIG. 6. Similarly, the fluid column extends from port 307 through lumen 710 and out through orifice 714 in the alternate embodiment relative to FIG. 7. This fluid column extends outside of the catheter from port 307 through conventional saline drip tubing 901 to a conventional disposable pressure transducer 902. This transducer may be flushed through additional saline drip tubing 902 from saline reservoir 903. The output of the pressure transducer 902 is connected via a conventional electronic connection means to the electronic control circuit 201. The signal is then transmitted to data acquisition circuit 203 and computer 206 for processing.

The pressure transducer 901, in conjunction with the electronic control circuit 201, the data acquisition circuit 203 and the computer 206, may be used to determine intracranial pressure in the patient 100 by periodically halting the retroperfusion flow by stopping pumps 102, 110 and 114, and measuring the quiescent intrasinus pressure in the cerebral vein. The balloon may also need to be deflated by running only pump 1 10 forward, or pump 102 in reverse for a short period. The measured pressure with retroperfusion flow stopped should be less than about 10 mmHg in a normal person under normal conditions, with the level of the transducer 901 positioned such that it is substantially horizontal with the level of the tip of the catheter in the patient. If the transducer is higher than the tip of the catheter, then the reading will be higher by the amount of water column height of vertical displacement, or the reading will be lower if the transducer is positioned lower with respect to the tip of the catheter by the amount of water column height of negative vertical displacement. This measured cerebral venous pressure with the pump off approximates the intracranial pressure, which is a key indicator of cerebral edema. If edema is increasing, the intracranial pressure will increase. Under this condition, the magnitude of the retroperfusion flow must be decreased, or the flow must be halted for a period of time until the intracranial pressure, and the intrasinus pressure which is used to approximate the intracranial pressure decreases to safe levels of about 10 mmHg or less.

Periodically stopping the retroperfusion flow as described supra is also useful for determining whether the pressure transducer signal is viable or degraded. Increasing flow should be accompanied by a concomitant increase in the pressure signal, with the converse also being true. Stopping flow should result in a significant drop in the pressure signal. Furthermore, the quiescent pressure signal with flow stopped should indicate high-frequency content variations of greater than a 1 second time constant such as momentary maxima and momentary minima with variations in blood pressure, such as those cause by each heart beat, or other changes which may be correlated to conventional blood pressure measurement means. If any of the stated changes described supra are not observable on the pressure signal, then there may be an occluded catheter lumen 307 or pressure port orifice 614 or 714. Since measurement of correct pressure in the sinus where the catheter is placed is critical to the safety of the procedure, the method of periodically changing the retroperfusion flow rate and looking for appropriate changes in the pressure signal as described supra, or measuring the quiescent sinus pressure with the flow stopped and comparing it to a safe level as described supra is an important part of the method of application of this system. To correct an occluded pressure transducer, catheter lumen or port orifice, the operator may flush the fluid column through the transducer, lumen and orifice with suitable isotonic diluent, such as plasmalyte or a saline solution from reservoir 903.

If the pressure as measured in the sinus near the distal tip of the catheter exceeds a preset limit such as 25 mmHg, the retroperfusion flow rate is reduced until the pressure is no longer above 25 mmHg. During the time that pressure is over 25 mmHg, the system may provide an audible and visual alarm, and may be configured in software to stop flow, then restart flow once the pressure drops below the 25 mmHg limit at a lower flow rate than the rate just prior to the alarm. If the pressure is significantly below 25 mmHg, such as 10 mmHg, then the system may increase flow rate either automatically or with operator interaction until a limit such as 25 mmHg is reached. If the system automatically raises flow rate, it may be configured to look for a concomitant increase in sinus pressure, and finding no correlation, it may be configured to activate another audible and visual alarm, indicating that a rise in flow is not resulting in a rise in pressure, which may be indicative of a disconnection of the extracorporeal tubing 107 from the venous cannula 101.

Referring now to FIGS. 8, 8A and 8B concurrently, there is shown an external and cross sectional representation of catheter tip 800 to provide a superior sagittal sinus insertion as shown in FIG. 5.

Once again, catheter 850 is a double lumen tube with a small, oval pressure lumen 810 and a large semi-circular blood flow lumen 811. The catheter has a slight taper toward the distal end thereof for ease in insertion, among other advantages.

The primary blood exit port 815 is located at the distal tip of the catheter. Thus, blood can flow through lumen 811 directly into the superior sagittal sinus 505. The site of exit port 815 may be selected to provide optimum blood flow into the sinus.

The pressure lumen 810 is open to the exterior of the catheter via aperture 814 adjacent to but, typically, spaced slightly from the exit port 815 for the reasons discussed supra. The operation of pressure lumen 810 and pressure aperture 814 is similar to the operation of the counterpart components in the other catheter embodiments.

Thus, there is shown and described a unique design and concept of a method of cerebral retroperfusion and an apparatus for performing the method. Reperfusion injury is reduced and treatment is enhanced by three means, viz. (1) initial use of venous blood which has a lower oxygen content than arterial blood, (2) filtering harmful cytokines that are present during ischemia, and (3) removing positively charged calcium ions which are also elevated within ischemic tissues by use of a negatively-charged platinum electrode in the extracorporeal circuit . Subsequent oxygenation and cooling of the blood are also provided. While this description is directed to a particular embodiment, it is understood that those skilled in the art may conceive modifications and/or variations to the specific embodiments shown and described herein. Any such modifications or variations which fall within the purview of this description are intended to be included therein as well. It is understood that the description herein is intended to be illustrative only and is not intended to be limitative. Rather, the scope of the invention described herein is limited only by the claims appended hereto.

1. A retroperfusion system comprising,
a catheter adapted for insertion into the cerebral portion of a patient's anatomy for carrying blood of the patient,
said catheter having first and second lumens therethrough, and
an inflatable balloon disposed at the distal end of said catheter,
said inflatable balloon in communication with said first lumen in said catheter.

2. The system recited in claim 1 including,
a filtration system for filtering the blood of the patient,
said catheter connected to said filtration system.

3. The system recited in claim I wherein,
said catheter includes at least two apertures therethrough to establish communication between said inflatable balloon and said first lumen.

4. The system recited in claim 1 wherein,
said catheter includes an end opening adjacent to the distal end thereof which communicates with said first lumen.

5. The system recited in claim 4 wherein,
said catheter includes at least one aperture through the body thereof from said first lumen and which does not communicate with said inflatable balloon.

6. The system recited in claim 5 wherein,
said catheter includes a plurality of apertures through the body thereof from said first lumen on the proximal side of said inflatable balloon.

7. The system recited in claim 3 wherein,
said two apertures define a ratio of the area of the respective openings thereof to establish a non-stagnating flow in said balloon.

8. The system recited in claim 1 including,
an introducer for inserting said catheter into the patient's anatomy.

9. The system recited in claim 8 wherein,
said introducer has a side port which is connected to deliver venous blood from the patient.

10. The system recited in claim 5 wherein,
said aperture is disposed in proximity to said inflatable balloon to enable fluid from said lumen to egress the catheter.

11. The system recited in claim 1 wherein,
said second lumen includes at least one opening to the exterior of said catheter located adjacent to said balloon.

12. The system recited in claim 11 wherein,
said second lumen is adapted to monitor pressure adjacent to the distal end of said catheter.

13. The system recited in claim 2 wherein,
said filtration system includes a hemoconcentrator filter connected in series with said catheter in order to filter any blood flow through said catheter.

14. The system recited in claim 13 wherein,
said filtration system includes a cytokine filter connected to said hemoconcentrator filter to filter a portion of a first effluent from said filtration system and to return a second effluent from said cytokine filter to said hemoconcentrator filter.

15. A retroperfusion system comprising,
a catheter adapted for insertion into the superior sagittal sinus portion of a patient's anatomy,
said catheter having first and second lumens therethrough,
said first lumen adapted to carry fluid flow therethrough,
said second lumen adapted to monitor the pressure in said superior sagittal sinus of a patient,
pump means for selectively pumping fluid through said first lumen,
at least one opening disposed adjacent to the distal end of said catheter and in communication with said first lumen in said catheter whereby fluid may flow from said catheter into said superior sagittal sinus, and
at least one opening disposed adjacent to the distal end of said catheter and in communication with said second lumen in said catheter whereby fluid pressure in said superior sagittal sinus can be monitored.

16. The system recited in claim 15 wherein,
said catheter has an outer diameter which is selected to at least partially occlude the superior sagittal sinus of the patient to restrict retrograde fluid flow from the superior sagittal sinus of the patient.

17. The system recited in claim 15 including,
feedback control means for sensing the pressure in said second lumen to control the flow of fluid through said first lumen into the superior sagittal sinus of the patient.

18. The system recited in claim 1 wherein,
said first lumen comprises a substantial portion of the cross-section of said catheter, and
said second lumen comprises a relatively small portion of the cross-section of said catheter.

19. The system recited in claim 3 wherein,
said first lumen carries a fluid flow therethrough and said fluid flows from said first lumen into said balloon via one of said apertures and from said balloon into said first lumen via another one of said apertures.

20. The system recited in claim 19 wherein,
said balloon is inflated by fluid flow therein through said two apertures.

21. The system recited in claim 11 wherein,
said balloon is inflated by fluid flow therein through said two apertures.

22. The system recited in claim 11 wherein,
said one opening is distal to said balloon.

23. The system recited in claim 2 wherein,
said filtration system includes oxygenator means for adding oxygen to the blood of the patient.

24. The system recited in claim 2 wherein,
said filtration system includes heat exchanger means for controlling the temperature of the blood of the patient.

25. The system recited in claim 15 including,
an introducer,
said introducer including a hollow tube for insertion into a vein of a patient and a hollow side port,
said hollow tube adapted to receive and guide said catheter into the patient's anatomy,
said hollow side port adapted to receive and guide venous blood flow from the patient,
said catheter and said side port adapted to be connected to an extracorporeal circuit for the patient's blood.

26. The system recited in claim 25 wherein,
said extracorporeal circuit includes,
pump means for causing blood to flow in said catheter, and
filter means for filtering the blood in said catheter.

27. The system recited in claim 15 including,
an inflatable balloon at the distal end of said catheter.

28. The system recited in claim 27 wherein,
said catheter includes at least one opening therein which communicates with said balloon.

29. The system recited in claim 1 including,
an electronic control connected to control the flow of blood of the patient in said catheter.

30. The system recited in claim 13 including,
a diluent source connected to said filtration source and operative to supply a diluent to the blood of the patient.

31. The system recited in claim 2 including,
a platinum electrode connected to said filtration system to remove charged ions from the blood of the patient.

32. The system recited in claim 30 including,
a load cell to measure the amount of diluent in said diluent source.

33. A method of retroperfusing an ischemic cranial region with venous blood comprising the steps of
inserting a catheter into a patient,
at least partially inflating a balloon affixed to the distal end of said catheter by passing the venous blood into said balloon via at least one aperture in said catheter, and
causing the flow of the venous blood from the patient through said catheter into the superior sagittal sinus of the patient in a retrograde flow direction.

34. The method recited in claim 33 wherein,
the flow of venous blood is supplied to said superior sagittal sinus via an opening at the end of said catheter distal to said balloon which is inserted into the superior sagittal sinus.

35. The method recited in claim 33 wherein,
the flow of venous blood is supplied to said superior sagittal sinus via an opening in said catheter proximal to said balloon which is inserted into a transverse sinus.

36. The method recited in claim 33 wherein,
said catheter has a first lumen for carrying the flow of venous blood therethrough, and
a second lumen for monitoring the pressure of the flow of venous blood adjacent to said catheter.

37. The method recited in claim 33 wherein,
the catheter is inserted percutaneously into an accessible vein of the patient.

38. A method of retroperfusing an ischemic cranial region comprising of the steps of:
inserting a double-lumen catheter into a vein of a patient through an introducer with a side port,
advancing said catheter into the superior sagittal sinus from said venous entry point through a sigmoid sinus and through a transverse sinus,
causing the flow of venous blood from the patient via the side port of the introducer through an extra-corporeal filtration system into a first of said lumens in said double-lumen catheter in a retrograde flow direction,
at least partially occluding the superior sagittal sinus with said catheter to increase the pressure in the superior sagittal sinus and to force at least some of said retrograde flow to progress retrogradely into the superior sagittal sinus,
sensing the pressure of the retrograde with the second lumen of said double-lumen catheter within the superior sagittal sinus, and
causing an increase or decrease in said retrograde flow in said first lumen based on the pressure sensed in the second lumen.

39. The method of cerebral retroperfusion recited in claim 38 including the step of;
monitoring intracranial pressure by temporarily halting the retroperfusion flow and measuring the quiescent intrasinus pressure.

40. The method recited in claim 39 including the step of;
testing the validity of the pressure signal by increasing the retroperfusion flow rate and checking for a concomitant increase in the pressure signal.

41. The method recited in claim 39 including the step of;
testing the validity of the pressure signal by decreasing the retroperfusion flow rate and checking for a concomitant decrease in the pressure signal.

42. The method recited in claim 39 including the step of;
testing the validity of the pressure signal by looking for local maxima and minima in the pressure reading of greater than 1 second time constant in correlation with changes in blood pressure of the patient.

43. The method recited in claim 40 including the step of;
clearing any unwanted occlusion in the fluid path connected to the pressure transducer by flushing said transducer with a saline solution.

44. The method recited in claim 38 wherein,
said transverse sinus is the right transverse sinus, and
said sigmoid sinus is the right sigmoid sinus.

45. The method recited in claim 38 wherein,
said double-lumen catheter is inserted into a jugular vein of the patient.

46. The method recited in claim 38 wherein,
said double lumen catheter is inserted into a femoral vein.

47. The system recited in claim 2 wherein,
said filtration system includes a neutrophil filter for removing neutrophils from the blood of the patient.

48. The system recited in claim 30 wherein,
said diluent is supplied to the blood of the patient to reduce the hematocrit of the blood of the patient by about 50% of normal.

49. The method recited in claim 37 wherein,
the catheter is inserted into the vein through a hollow introducer which is inserted percutaneously into the accessible vein.

50. The method recited in claim 49 wherein,
said hollow introducer includes a port through which the patient's venous blood can be extracted from the accessible vein.

51. The method recited in claim 50 wherein, the patient's venous blood extracted via the port of said introducer is supplied to the catheter.

52. The method recited in claim 51 wherein,
the patient's venous blood is passed through a filtration system after extraction from the patient and before being supplied to the catheter.

53. A method of retroperfusing the cranial anatomy of a patient comprising the steps of,
inserting a hollow introducer having a side port into a vein of a patient whereby venous blood can be extracted from the patient via the side port,
inserting a two-lumen catheter into the vein of the patient via the hollow introducer,
advancing the catheter into the cranial anatomy of the patient to a position adjacent to the superior sagittal sinus of the patient,
pumping the patient's venous blood from the side port of the introducer to the superior sagittal sinus of the patient one of the lumens in the catheter, and
monitoring the pressure of the venous blood at the superior sagittal sinus via the other lumen in the catheter.

54. The method recited in claim 53 including the steps of:
selectively inflating and deflating a portion of the two-lumen catheter to at least partially occlude vessels in the cranial anatomy of the patient in order to control the pressure of the venous blood which is being pumped into the superior sagittal sinus of the patient.

55. The method recited in claim 53 including the step of:
filtering the venous blood pumped from the side port of the introducer to the one lumen of the catheter.
